(19) United States
(12) Patent Application Publication
(10) Pub. No.: US 2026/0031206 A1
(43) Pub. Date: Jan. 29, 2026

(54) MEDICAL DATA MANAGEMENT APPARATUS, MEDICAL DATA MANAGEMENT PROGRAM, AND MEDICAL DATA MANAGEMENT METHOD

(71) Applicant: FUJIFILM Corporation, Tokyo (JP)

(72) Inventor: Takanobu OTSUBO, Tokyo (JP)

(73) Assignee: FUJIFILM Corporation, Tokyo (JP)

(21) Appl. No.: 19/347,718

(22) Filed: Oct. 2, 2025

Related U.S. Application Data
(63) Continuation of application No. PCT/JP2024/012711, filed on Mar. 28, 2024.

(30) Foreign Application Priority Data
Apr. 5, 2023 (JP) 2023-061527

(51) Int. Cl. G16H 10/60

(52) U.S. Cl. CPC G16H 10/60

(57) ABSTRACT

A medical data management apparatus according to the present invention, including a processor, in which the processor is configured to: perform a first editing process of editing, in medical data including personal information capable of identifying an individual and health data related to health of the individual, an item capable of identifying the individual from the personal information; and perform a second editing process of editing medical data after the first editing process in accordance with an editing level set according to affiliation information of an output destination and an intended use.

### CROSS-REFERENCE TO RELATED APPLICATIONS

This application is a Continuation of PCT International Application No. PCT/JP2024/012711 filed on 28 Mar. 2024, which claims priority under 35 U.S.C § 119 (a) to Japanese Patent Application No. 2023-061527 filed on 5 Apr. 2023. The above application is hereby expressly incorporated by reference, in its entirety, into the present application.

### BACKGROUND OF THE INVENTION

### 1. Field of the Invention

The present invention relates to a medical data management apparatus, a non-transitory computer readable medium storing a medical data management program, and a medical data management method.

### 2. Description of the Related Art

In a case where a medical examince for medical examination or a patient in a medical institution agrees to utilize medical data of the medical examince or the patient for health of the medical examinee or the patient or for contribution or the like to industry, the medical institution can utilize the medical data of the individual according to the personal information protection laws of each country. Each medical examination center or medical institution manages personal medical information in a form in which an individual can be identified, and in a case where the personal medical information is to be provided to a third party such as a research institution, a university, a pharmaceutical company, or an insurance company, it is necessary to pseudonymize or anonymize the personal medical information based on consent of the individual such that the individual cannot be identified by the data alone, based on the personal information protection law or the like.

In JP2016-048530A, in a case where storage or management of digital medical image data including personal information is outsourced externally, in a medical institution, the personal information of the medical image data is protected. Specifically, it is described that three steps of an anonymization process, an encryption process, and a secret distribution process are executed on the image data to which the personal information of a patient is added as metadata, and then the image data is transmitted to a data center outside the medical institution.

### SUMMARY OF THE INVENTION

On the other hand, an editing content of the personal information to be requested varies depending on a type and a use of the data, the laws and regulations for corresponding personal information protection, and the like in the provision of the personal information, such as health data of an individual in the medical institution, to the third party. The editing content is determined according to an intended use of the personal information or information such as a country to which the personal information is to be crossed over, but in the editing process of the related art, information on the required editing content cannot be grasped, and an intended use of the personal information is not advanced.

Information obtained by editing the personal information, for example, information obtained by performing a pseudonymization process, varies depending on the laws and regulations of each country, such as whether the information is allowed to be transferred to a third party or is limited to the analysis use within the facility. In addition, it is assumed that there is a risk that information that is not necessary for the data user is included, and there is a risk from the viewpoint of ensuring the safety of personal information. Therefore, it is required to enhance the safety of the personal information and to perform editing of the medical data in accordance with the intention of the data user.

An object of the present invention is to provide a medical data management apparatus, a non-transitory computer readable medium storing a medical data management program, and a medical data management method that enable data utilization in accordance with the laws and regulations of each country, safety of personal information, or an intention of a data user.

According to an aspect of the present invention, there is provided a medical data management apparatus comprising a processor, in which the processor is configured to: perform a first editing process of editing, in medical data including personal information and health data related to health of an individual, an item capable of identifying the individual from the personal information; and perform a second editing process of editing medical data after the first editing process in accordance with an editing level according to an output destination.

It is preferable that the medical data after the first editing process is restorable to the medical data before the first editing process, and the editing level is a level at which medical data after the second editing process is restorable to medical data before the second editing process.

It is preferable that in a case where the second editing process is performed on the medical data after the first editing process at the editing level at which the medical data is restorable, the medical data is capable of identifying the individual by combining a plurality of pieces of health data after the second editing process.

It is preferable that the editing level is a level at which an item having a low priority in the restorable medical data after the second editing process is information having a lower accuracy than an accuracy of the item in the medical data before the second editing process.

It is preferable that the medical data after the first editing process is restorable to the medical data before the first editing process, and the editing level is a level at which medical data after the second editing process is non-restorable to medical data before the second editing process.

It is preferable that the editing level is a level at which an item having a low priority in the non-restorable medical data after the second editing process is information having a lower accuracy than an accuracy of the item in the medical data before the second editing process.

It is preferable that the editing level is set with reference to a conversion table in which the output destination and the editing level are associated with each other.

It is preferable that the conversion table is set or updated by a user interface.

It is preferable that the editing level is switched between a restorable level and a non restorable level according to an intended use of the output destination.

It is preferable that in a case where the editing level in a state in which the intended use is the same and for a relationship between an output source and the output destination of the medical data, the output source and the output destination are in the same country is set as a standard editing level, the editing level is set to be higher than the standard editing level in a state in which the output source and the output destination are in countries different from each other; and the editing level is set to be lower than the standard editing level in a state in which the output source and the output destination are in the same country and are related institutions.

It is preferable that in the second editing process, an accuracy reducing process of reducing an accuracy of information is performed, for an item indicating a rare disease.

It is preferable that in a case where the medical data includes a medical image, the personal information includes personal identification information determined for each item and personal image information for identifying the individual from the medical image.

According to another aspect of the present invention, there is provided a non-transitory computer readable medium storing a medical data management program causing a computer to execute: a function of a first editing process of editing, in medical data including personal information and health data related to health of an individual, an item capable of identifying the individual from the personal information; and a function of a second editing process of editing medical data after the first editing process in accordance with an editing level according to an output destination.

According to still another aspect of the present invention, there is provided a medical data management method comprising: a step of a first editing process of editing, in medical data including personal information and health data related to health of an individual, an item capable of identifying the individual from the personal information; and a step of a second editing process of editing medical data after the first editing process in accordance with an editing level according to an output destination.

It is preferable that a facility that performs the first editing process is a facility on a provision side of the medical data, and a facility that performs the second editing process is a facility on a use side of the medical data, and the method further comprises: a step of transmitting the medical data after the first editing process and the medical data before the second editing process from a server of the facility on the provision side of the medical data to a server of the facility on the use side of the medical data.

According to the present invention, it is possible to utilize data in accordance with the laws and regulations of each country, safety of personal information, and an intention of a data user.

### BRIEF DESCRIPTION OF THE DRAWINGS

FIG. 1 is a schematic diagram of a medical data management system.

FIG. 2 is a block diagram illustrating a function of a medical data management apparatus.

FIG. 3 is an explanatory diagram of a first editing process and a second editing process on medical data.

FIG. 4 is an explanatory diagram for pseudonymization on personally identifiable information in the first editing process.

FIG. 5 is an explanatory diagram of a second editing process at a first level.

FIG. 6 is an explanatory diagram of the second editing process at a second level.

FIG. 7 is an explanatory diagram of the second editing process at a third level.

FIG. 8 is an explanatory diagram of the second editing process at a fourth level.

FIG. 9 is an explanatory diagram of receiving a command operation via a user interface.

FIG. 10 is an explanatory diagram of anonymization of personal identification information included in a medical image.

FIG. 11 is a flowchart illustrating a series of flows of the first editing process and the second editing process performed on the medical data.

FIG. 12 is a block diagram illustrating a function of a medical data provision apparatus and a medical data using apparatus according to a second embodiment.

FIG. 13 is a schematic diagram of a medical data management system according to a modification example of the second embodiment.

### DESCRIPTION OF THE PREFERRED EMBODIMENTS

### First Embodiment

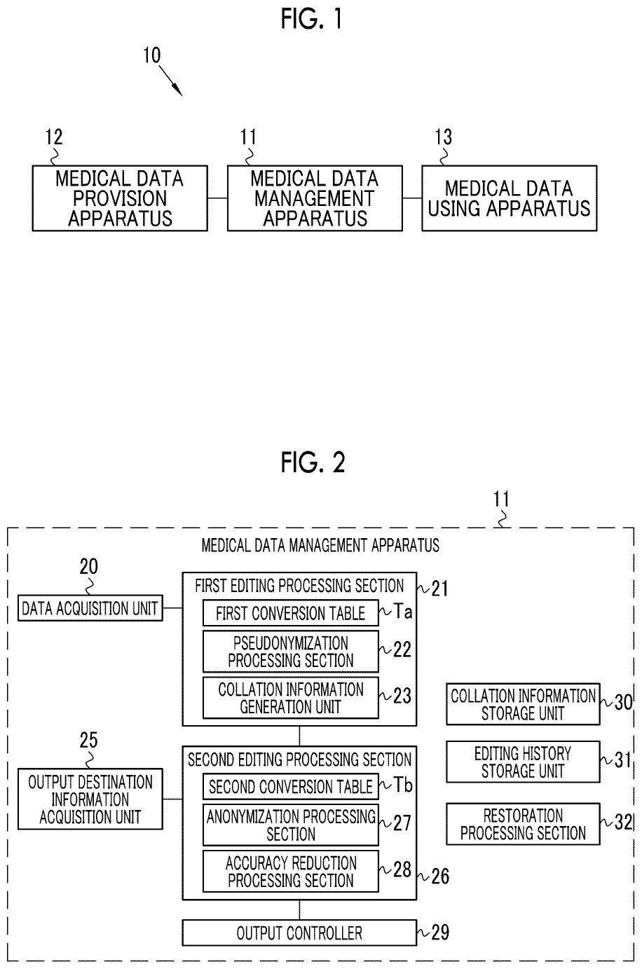

As illustrated in FIG. 1, a medical data management system 10 is configured with a medical data management apparatus 11, a medical data provision apparatus 12 that provides medical data to the medical data management apparatus 11, and a medical data using apparatus 13 that is used by a data user to use the medical data output from the medical data management apparatus 11. The medical data management apparatus 11 is an apparatus that can transmit and receive medical data and includes a storage medium and a processor, and performs an editing process by detecting personal information such as a name or an address for the received medical data. The medical data management apparatus 11, the medical data provision apparatus 12, and the medical data using apparatus 13 are managed by constituent organizations having independent authorities from each other.

The medical data provision apparatus 12 is a hospital, a medical device management company, or the like, and provides medical data of a medical examinee or a customer collected by an examination of a hospital or the like to the medical data using apparatus 13 which is a third party, via the medical data management apparatus 11 that performs the editing process. The medical data using apparatus 13 allows the data user, such as a facility or a company, to utilize the edited medical data for each use. It is preferable that the medical data management apparatus 11 is managed by a certified anonymization editing medical information creation business operator (certified business operator) that receives a commission for anonymization editing from a medical institution or the like having the medical data provision apparatus 12. In addition, the medical data provision apparatus 12 and the medical data management apparatus 11 communicate medical data having personal information through a dedicated line separated from the open network.

As illustrated in FIG. 2, the medical data management apparatus 11 implements functions of a data acquisition unit 20, a first editing processing section 21, an output destination information acquisition unit 25, a second editing processing section 26, an output controller 29, a collation information storage unit 30, an editing history storage unit 31, and a restoration processing section 32. In addition, the first editing processing section 21 includes a first conversion table Ta, a pseudonymization processing section 22, and a collation information generation unit 23, and the second editing processing section 26 includes a second conversion table Tb, an anonymization processing section 27, and an accuracy reduction processing section 28. The medical data management apparatus 11 is a computer, such as a personal computer or a workstation, in which each application program for implementing a predetermined function is installed. The computer includes a central processing unit (CPU) which is a processor, a memory, a storage, and the like, and implements various functions by a program or the like stored in a storage.

By using the above functions, the medical data management apparatus 11 performs editing of medical data in accordance with the laws and regulations of each country, the safety of personal information, and an intention of a data user, and provides the edited medical data. In addition, the medical data management apparatus 11 also has a function of an input receiving unit (not illustrated), and an input of a user who is an administrator or the like of an organization to which the medical data management apparatus 11 belongs can be received by an operation via a user interface (UI) such as a mouse operation or a keyboard operation. The input includes a control of an editing level and an instruction of an individual editing process for each item.

The medical data includes personal information such as personal identification information that is an item for identifying or identifying an individual, and health data related to the health of the individual. The health data is data including biological data such as medical images measured at a hospital or a medical examination center, a finding of medical examination such as a blood test, and diagnostic information, and the personal identification information is information such as a name, a gender, a date of birth, a medical history, a medication history, a family history, a hospital visit history, an address, an email address, and a medical examinee ID. The personal information refers to all information that can identify an individual by a combination of the personal identification information, personal image information such as a rare region included in a medical image, a rare value in health data, a doctor's opinion, and the like. The personal information included in the medical data and the health data of the individual are information on the same person.

The data acquisition unit 20 acquires personally identifiable medical data, which is medical data including health data and personal information, from the medical data provision apparatus 12. The acquired personally identifiable medical data is transmitted to the first editing processing section 21, and a pseudonymization process is performed.

The first editing processing section 21 performs a first editing process, which is a pseudonymization process, on an item that can identify an individual from the personal information in the received personally identifiable medical data. The first editing processing section 21 includes the first conversion table Ta for recognizing personal identification information, the pseudonymization processing section 22 that performs pseudonymization of converting the personal identification information into a pseudonym ID to generate pseudonym medical data, and the collation information generation unit 23 that generates individual collation information to be used for collation between the pseudonym ID and each piece of personal identification information, in association with the generation of the pseudonym ID.

The first conversion table Ta is for recognizing the personal identification information which is an editing target, and determining a content of the first editing process of substituting the personal identification information with the pseudonym ID or the like. A character, a character string, or a symbol after the substitution may be determined according to a pattern registered in advance according to the number or a type of the personal identification information to be substituted. In addition, it is preferable to perform pseudonymization with the minimum editing content, such as not only substitution for the pseudonym ID but also leaving information on an address, an age, and a medical history with a decrease in accuracy to the extent that the individual cannot be identified.

The pseudonymization in the pseudonymization processing section 22 is an editing process of generating pseudonym medical data in which a character string of personal information in personally identifiable medical data is edited and with which a specific individual cannot be identified unless the edited character string is collated with other information. On the other hand, in order to set the personally identifiable medical data to a state in which the pseudonym medical data is restorable as necessary, the pseudonym ID is a character string that does not overlap with a plurality of other pseudonym IDs. The generated pseudonym medical data is transmitted to the second editing processing section 26. The pseudonymization information is stored in a region other than the collation information storage unit 30. In addition, in a case where there is information for identifying an individual in the health data, it is preferable to perform a substitution or an accuracy reducing process.

The collation information generation unit 23 generates individual collation information to be used for restoration from the pseudonym medical data to personally identifiable medical data. By collating the pseudonym medical data with the individual collation information, the pseudonym medical data, which is medical data after the first editing process, is restorable to the personally identifiable medical data, which is medical data before the first editing process. The individual collation information is stored in the collation information storage unit 30.

The output destination information acquisition unit 25 acquires affiliation information of the medical data using apparatus 13 and information on an intended use of the medical data as output destination information. The affiliation information includes at least information on an affiliation country and an affiliation corporation, and the specific output destination is a medical institution, a pharmaceutical company, a research institution, an administrative agency, or the like. The intended use is “research”, “machine learning”, “analysis for medical treatment”, “analysis”, “medication data analysis”, “insurance plan creation”, “health advice”, “clinical trial recruiting”, or the like. The affiliation information can be classified with whether or not a relationship with an output source is the same country or corporation, and for example, an editing level is increased in an order of “the same country and the same corporation”, “the same country”, and “another country” in the affiliation information.

The second editing processing section 26 performs a second editing process on the pseudonym medical data acquired from the first editing processing section 21, according to the editing level set according to the output destination information. It is preferable that the editing level is set by a pattern registered in advance in the second conversion table Tb. The second editing processing section 26 includes the second conversion table Tb, the anonymization processing section 27 that performs an anonymization process of performing anonymization, and the accuracy reduction processing section 28 that reduces an accuracy of health data. The anonymous medical data in which the personal information is anonymized can be provided to a third party without consent of an individual. The second editing process executed according to the output destination information includes not performing the editing.

In the second conversion table Tb, an editing content including an editing level in the second editing process is set based on pattern information registered in advance according to the output destination information, a type of the medical data as an editing target, and an accuracy of the data. In addition, an item that causes the accuracy to be decreased, which includes a distribution of a priority of each item in the health data, is also determined. It is preferable that the pattern information is obtained by using a content of the second editing process executed in the past.

In the anonymization in the anonymization processing section 27, pseudonym information having at least the pseudonym ID is converted into an anonymous ID which is a different character string, and anonymous health data having anonymous information which is edited such that a specific individual cannot be identified is generated. In addition, for each piece of health data, a mask process of masking by substituting with a meaningless character or character string, blacking out, or the like is performed as necessary, and unnecessary data is deleted. The anonymous information is in a state in which the anonymous information cannot be restored to pseudonym information.

In the accuracy reduction processing section 28, the accuracy reducing process of reducing an accuracy of an examination value or the like for an item distributed as having a low priority is performed with the second conversion table Tb, and each piece of health data constituting pseudonym medical data 43 is converted into the edited health data. For example, a numerical value of the examination value is rounded to one or two significant figures by rounding off, truncating a decimal point, or the like, and the examination value is converted into a stepwise evaluation of a position of the examination value with respect to a normal value range.

The output controller 29 outputs the medical data on which the first editing process and the second editing process are performed, to the medical data using apparatus 13. In addition, the edited medical data may be displayed on a display (not illustrated) as necessary. The display may be performed not only for the result of the second editing process but also for the result of the first editing process.

The collation information storage unit 30 stores the individual collation information generated by the collation information generation unit 23. The individual collation information is identified based on the pseudonym ID or the like in a case where a restoration process of the pseudonym medical data is performed. The pseudonym medical data and the individual collation information are stored in regions different from each other not to lead to the identifying of the individual.

The editing history storage unit 31 stores, as editing history data, at least any one of a first editing history indicating that the first editing process has been performed or a second editing history indicating that the second editing process has been performed.

The restoration processing section 32 performs the restoration process of restoring (reverse-converting) the pseudonym medical data to the personally identifiable medical data. In the restoration process, individual collation information stored in the collation information storage unit 30 is identified based on the pseudonym ID of the pseudonym medical data to be restored, and the pseudonymous medical data is collated from the medical examinee ID or the like of the personally identifiable medical data of the individual collation information. The restoration process is executed, for example, in a case where the pseudonym medical data is used for the use of performing “health advice” or “clinical trial recruitment” in the medical data using apparatus 13, in order to provide a feedback on the obtained result to the medical examinee. A specific storage location of the personally identifiable medical data is not limited as long as the personally identifiable medical data is stored in a region different from the collation information storage unit 30.

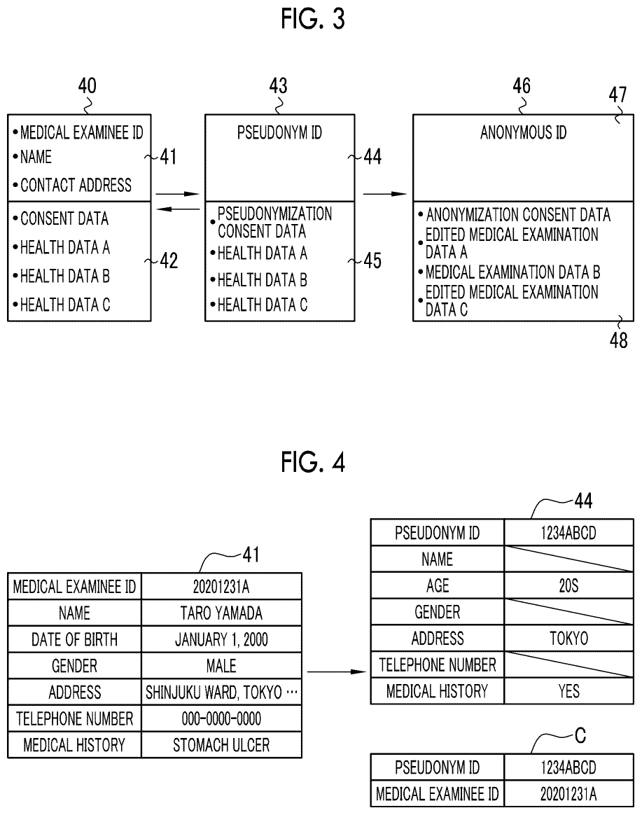

As illustrated in FIG. 3, in a case where anonymization of medical data is performed by the medical data management apparatus 11, the personally identifiable medical data 40 is converted into the pseudonym medical data 43 by the first editing process, and the pseudonym medical data 43 is converted into anonymous medical data 46 by the second editing process. The personally identifiable medical data 40 has personally identifiable information 41 and personally identifiable health data 42 having personal identification information such as a medical examinee ID and a name, which are input in a medical institution. The personally identifiable health data 42 in the personally identifiable medical data 40 includes consent data, in addition to each piece of health data such as biological data.

The pseudonym medical data 43 includes pseudonym information 44 in which the personally identifiable information 41 is pseudonymized and pseudonym health data 45 in which at least the consent data is pseudonymized from the personally identifiable health data 42. The anonymous medical data 46 includes anonymous information 47 in which the pseudonym information 44 is anonymized and anonymous health data 48 in which at least pseudonymization consent data of the pseudonym health data 45 is anonymized.

The consent data is data indicating consent of a medical examinee regarding the use of the medical data, and includes a signature or the like of the medical examince, and thus has personal identification information. Therefore, in a case of pseudonymization or anonymization, the personal identification information of the consent data is also edited.

As illustrated in FIG. 4, in pseudonymization, which is the first editing process, each item that is personal identification information in the personally identifiable information 41 is replaced or an accuracy is reduced to generate the pseudonym information 44 having at least a pseudonym ID. For example, in a case where the personally identifiable information 41 has information on a medical examinee ID, a name, a date of birth, a gender, an address, a phone number, and a medical history, the medical examinee ID, the name, the gender, and the phone number are deleted in a case of generating the pseudonym ID, and the accuracy of the date of birth, the address, and the medical history is reduced. The date of birth is converted into a rough age group such as “20s”, the address is converted into a division of a local government such as “Tokyo”, and the medical history is converted into “Yes”.

In addition, in the pseudonymization, individual collation information C is also generated. The individual collation information C is, for example, information in which the medical examinee ID and the pseudonym ID are associated with each other, or information in which the personally identifiable medical data 40 other than the medical examinee ID and the pseudonym ID are associated with each other.

In the second editing process, the anonymization of converting the pseudonym information 44 having the pseudonym ID in the restorable state into the anonymous ID in a non-restorable (non-reversible) state and the accuracy reducing process of reducing an accuracy of an examination value of the health data are executed according to an editing level. The editing level for setting whether or not to execute the anonymization and the accuracy reducing process is switched depending on the affiliation information or the intended use.

In the second editing process, a priority according to an output destination information is set for each item of the health data by the second conversion table Tb, and the accuracy reducing process or deletion is performed according to the priority. The priority is set to four levels of “high”, “medium”, “low”, and “unnecessary” according to the output destination information, for example. In “medium”, editing is not performed, in “low”, an accuracy reducing process is performed, and in “unnecessary”, deletion is performed. In a case of “high”, in addition to not being edited, highlight display or protection from editing may be performed in a case of screen display. An item set to “low”, that is, a content of the accuracy reducing process will be described below.

The editing level is set to any of the following first to fourth levels obtained by combining whether to execute the anonymization with which the pseudonym medical data 43 is non-restorable to the personally identifiable medical data 40 or the accuracy reducing process of reducing the accuracy of the health data, in the second editing process performed on the pseudonym medical data 43.

The editing level is determined according to affiliation information of an output destination and an intended use of the output destination. For example, in a case where the intended use is to feedback a use result to a medical examinee, such as “health advice”, “clinical trial recruiting”, and “analysis for medical treatment”, the editing level is set to the first level or the second level, which is a restorable level, and in a case where the intended use is not to feedback the use result to the medical examinee, such as “machine learning”, “research”, “statistics”, and “analysis”, the editing level is set to the third level or the fourth level, which is a non-restorable level.

In a case where the intended use is “health advice” or “clinical trial recruiting”, the priority is low since an accurate examination value is not required in the entire health data. For example, it is sufficient that it is possible to discriminate whether the examination value measured in the medical examination falls within the normal value range or is higher or lower than the normal value range, and the accuracy reducing process rounds the examination value to a value of about 1 or 2 significant figures by performing rounding, rounding down, or the like.

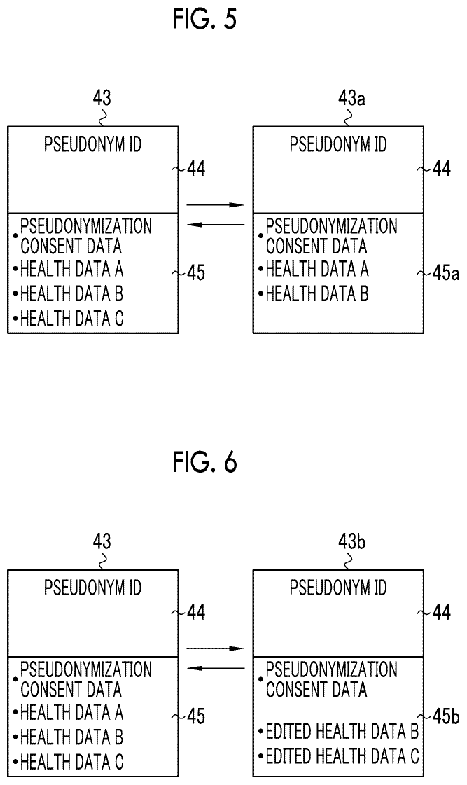

In the second editing process of the first level, the medical data is converted into medical data of “restorable and accuracy-maintained” on which the anonymization and the accuracy reducing process are not executed. In the second editing process of the second level, the medical data is converted into medical data of “restorable and accuracy-reduced” on which only the accuracy reducing process is executed. In the second editing process of the third level, the medical data is converted into medical data of “non-restorable and accuracy-maintained” on which only the anonymization is executed. In the second editing process of the fourth level, the medical data is converted into medical data of “non-restorable and accuracy-reduced” on which the anonymization and the accuracy reducing process are performed. In addition, an unnecessary item may be deleted as appropriate in any of the first to fourth levels. Hereinafter, examples of the first to fourth editing levels implemented by a combination of each intended use and affiliation information will be described with reference to FIGS. 5 to 8.

In a case where the second editing process is performed at the first level or the second level in a restorable state, the medical data after the second editing process can be used to identify an individual by being combined with a plurality of pieces of health data after the second editing process or by being collated by using the individual collation information C.

The first level is set in a case where the output destination is “the same country and the same corporation” and an accurate examination value is required for “analysis for medical treatment” of the intended use, for example. The second level is set in a case where the output destination is “same country” and the intended use is “health advice” and the medical data is fed back, while the examination value of the health data can be used even in a case where the examination value is not an accurate value. The first level and the second level are restorable levels at which pseudonym medical data 43a, which is medical data after the second editing process, can be restored to the pseudonym medical data 43, which is medical data before the second editing process.

As illustrated in FIG. 5, in the second editing process of the first level, editing of the health data is performed without performing the anonymization and the accuracy reducing process on the pseudonym medical data 43, and the pseudonym medical data 43 is converted into the pseudonym medical data 43a. Specifically, unnecessary health data in the pseudonym health data 45 is deleted according to the intended use or the like of the output destination, and the pseudonym health data 45 is edited into pseudonym health data 45a. As a result, it is possible to provide the medical data in accordance with an intention of a data user while reducing a data capacity. The pseudonym medical data 43 may be output as it is without performing editing such as deletion of unnecessary data, and even in this case, it is preferable to treat the second editing process as being executed.

For example, the second editing process of the first level is implemented in a case where the affiliation information is “same country and same corporation” and the intended use is “analysis for medical treatment”. In order to use the medical data for the treatment of the medical examince, the health data is analyzed in a different department of the same medical institution. The analysis is, for example, image interpretation by a specialist or image analysis using a medical device, and the medical data using apparatus 13 is a computer or the like in a different department. Since the medical data is used in the same hospital, in the second editing, the pseudonym medical data 43 having the pseudonym health data 45a in which only unnecessary data for a use is deleted is generated.

As illustrated in FIG. 6, in the second editing process of the second level, the accuracy reducing process of reducing an accuracy of information having a low priority in the pseudonym health data 45 in the pseudonym medical data 43 is performed to convert the pseudonym medical data 43 into the pseudonym medical data 43b. Specifically, the accuracy reducing process or the deletion of unnecessary data is performed on each piece of health data in the pseudonym health data 45 according to an intended use or the like of an output destination, and edits the health data into pseudonym health data 45b. As a result, it is possible to provide the medical data in accordance with an intention of a data user while maintaining the accurate health data.

For example, the second editing process of the second level is implemented in a case where the affiliation information is “same country and same corporation” and the intended use is “health advice”. In order to use the medical data for creating advice contents for improving a lifestyle of the medical examinee or the like, that is, for a feedback, analysis of the health data is performed in another department or the like of the same corporation, which is providing the data without anonymization. The analysis is, for example, image interpretation by a specialist or image analysis using a medical device, and the medical data using apparatus 13 is a computer or the like in a different department. Since the health data is used in the same hospital, in the second editing, the pseudonym health data 45b in which unnecessary data is deleted and the accuracy reducing process of the data to be used is performed is generated as the pseudonym medical data 43.

In the third level, for example, in a case where the output destination is “same country and different corporation” or the like, non-personal information that can be used without consent of the individual is set. In the fourth level, for example, in a case where the output destination is “another country” or the like, the personal information is anonymized to be non-personal information. In a case where the medical data is set to the third level or the fourth level, the anonymous medical data 46, which is medical data after the second editing process, is in a non-restorable state in which the anonymous medical data 46 cannot be restored to the pseudonym medical data 43, which is medical data before the second editing process.

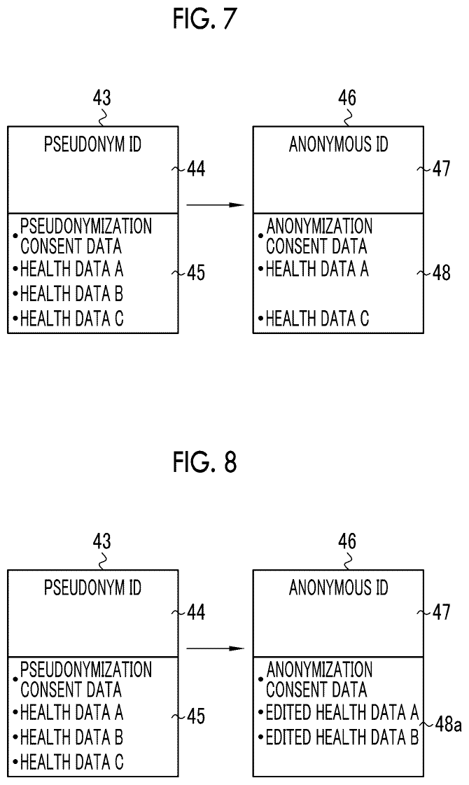

As illustrated in FIG. 7, in the second editing process of the third level, the anonymization is performed without performing the accuracy reducing process of the pseudonym medical data 43, and the pseudonym medical data 43 is converted into the anonymous medical data 46. Specifically, the pseudonym information 44 is converted into the anonymous information 47, and unnecessary data is deleted according to the intended use or the like of the output destination. As a result, it is possible to provide the medical data in accordance with the intention of the data user while protecting the personal information of the medical examinee and maintaining the accurate health data.

For example, the second editing process of the third level is implemented in a case where the affiliation information is “same country and different corporation” and the intended use is “machine learning”. In “machine learning”, the medical data is used in the medical data using apparatus 13 which is a computer of a corporation different from a hospital or a medical examination center that acquires the medical data, such as a medical device manufacturer. In the machine learning, since it is necessary to maintain the accuracy of the health data, the accuracy reducing process is not executed. Since the health data is used by different corporations and the personal identification information is not necessary information in the machine learning, the anonymous medical data 46 having the anonymized health data 48 in which unnecessary data is deleted and the anonymization is performed on the data to be used is generated in the second editing process.

As illustrated in FIG. 8, in the second editing process of the fourth level, the anonymization and the accuracy reducing process are performed to convert the pseudonym medical data 43 into anonymous medical data 46. Specifically, the anonymization and the accuracy reducing process or the deletion of unnecessary data are performed according to the intended use or the like of the output destination. As a result, it is possible to provide the minimum necessary medical data while protecting the personal information of the medical examine.

For example, the second editing process of the fourth level is implemented in a case where the affiliation information is “another country” and the intended use is “statistics”. In “statistics”, a large amount of medical data with less bias is used. Therefore, the medical data is used in, for example, the medical data using apparatus 13 which is a computer of a research institution in the other country. Since the personal identification information is not information necessary for machine learning and is required to be non-personal information based on the laws and regulations, in the second editing, the anonymous medical data 46 having the anonymous health data 48a on which the deletion of unnecessary data in use, anonymization, and the accuracy reducing process for the data is generated.

As illustrated in the use examples described above, the editing level is greatly affected by the affiliation information. Even in a case where the intended uses are the same, the editing level is changed according to a relationship between an institution that is an output source of the medical data and an institution that is an output destination. As the relationship between the output source institution and the output destination institution is closer, the editing level can be set lower. For example, in a case where the intended use is “for research”, the second editing level is set to a standard editing level in a case of “the same country and a different corporation”, the third level or the fourth level higher than the standard editing level is set in a case of “another country”, and the editing level is set to the first level lower than the standard editing level in a case of “the same country and the same corporation”. For example, in a case where the intended use is to provide a feedback, such as “health advice”, since the medical data is anonymized in a case where the output destination is “another country”, the pseudonym medical data 43 which is restorable by obtaining the consent of the medical examince separately is output, and it is preferable that the output destination of the third level or higher is excluded from a target. The output destination of the same corporation is an institution related to the institution of the output source.

In a case where an item of health data with which an individual can be identified alone or in combination, such as an examination value indicating a rare disease in the second editing process, is included, the accuracy reducing process is performed on the item. The accuracy reducing process may be individually performed after the first editing process or the second editing process, for example, editing in a step different from the setting of the priority or the editing level based on the output destination information. The rare disease is, for example, a disease with an incidence rate of less than 0.1% for the population, and a situation may occur in which only one case can be checked at the same medical institution. Therefore, an accuracy of the information indicating the rare disease is reduced, and the individual cannot be identified. In addition, in a case where the individual can be identified by combining the pseudonym information with the age group or the residence area, the accuracy reducing process is also performed on the pseudonym information 44. It is preferable to perform the accuracy reducing process on rare health data other than the rare disease, such as health data indicating a state of suffering from a plurality of diseases at the same time.

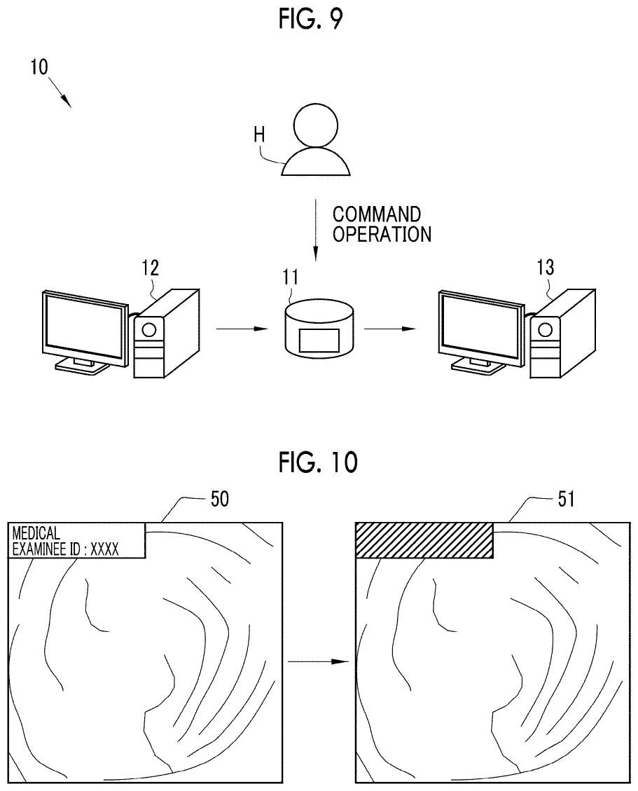

As illustrated in FIG. 9, the medical data management apparatus 11 receives an input of setting or updating of the first conversion table Ta and the second conversion table Tb by a command operation of an administrator H via a user interface. After the input, medical data is edited according to the changed conversion table. It is preferable that a content of the command operation is stored in the editing history storage unit 31. In addition, in a case where editing of a content that is not stored in the second conversion table, such as editing of health data related to a rare disease, is required, the administrator H may manually perform the second editing process. The administrator H is, for example, a substantial administrator or the like of the medical data management apparatus 11.

In a case where the second editing process is performed at a high frequency at the same editing level, such as the acquisition of the same output destination information, in using the medical data management apparatus 11, the standard editing level may be set as the editing level in the standard state. The high frequency is, for example, a case where the same editing level is set with a ratio of 50% or more from the editing history.

A case where the editing process is performed on a medical image in medical data will be described with reference to FIG. 10. Personal image information having biological data, such as an identifier which is personal identification information included in the medical image 50 or a characteristic case location or part in the medical image 50 that can identify an individual, is included in personal information, together with personal identification information determined for each item, such as a name or an address, and is required to be pseudonymized or anonymized. For example, the medical image 50 is edited into an anonymous medical image 51 in which the medical examinee ID, which is an identifier, is masked. In addition, even in a case where an individual cannot be identified only with the medical image 50, a feature portion that leads to the identifying of the individual is edited according to an editing level in combination with other information.

In the pseudonymization or the anonymization of the medical image 50, masking or deletion of the identifier included in the medical image 50, masking such as filling in the medical image 50, trimming processing of extracting a range that does not lead to the identifying of the individual in the medical image 50, deletion of the medical image 50 from the medical data, and the like are performed. In a case of the pseudonymization, it is preferable to replace a portion of the personal identification information with a pseudonym ID or the like.

Since the editing process on the medical image is highly difficult, a method of pseudonymization or anonymization on the medical image may be switched according to performance of the medical data management apparatus 11. For example, in a case where it is difficult to delete or mask only a feature portion in the medical image 50 or in a case where it takes time even in a case where it is possible to delete or mask only the feature portion, the medical image 50 itself may be deleted regardless of the content of the feature portion.

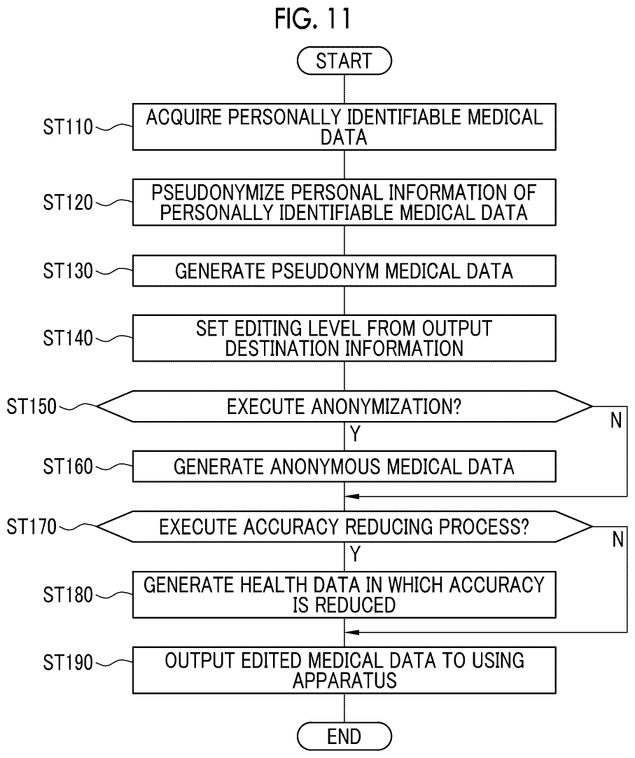

A series of flows of an operation in the editing process of the medical data management apparatus 11 according to the present embodiment will be described with reference to a flowchart illustrated in FIG. 11. The medical data management apparatus 11 acquires the personally identifiable medical data 40 including personal information and health data related to health of an individual from the medical data provision apparatus 12 (step ST110). A first editing process of recognizing the personal information from the acquired personally identifiable medical data 40 and pseudonymizing the personal information is performed (step ST120). In the first editing process, the individual collation information C obtained by associating personally identifiable information with a pseudonym ID and the pseudonym medical data 43 having pseudonym information are generated. The pseudonym medical data 43 is transmitted to the second editing processing section 26 (step ST130). An editing level in a second editing process is set from output destination information acquired from the medical data using apparatus 13 (step ST140). In the second editing processing section 26, anonymization on the pseudonym medical data 43 is performed according to the editing level (step ST150).

In a case where the editing level is set to a third level or higher (Y in step ST150), the anonymization is performed on the pseudonym medical data 43 to generate the anonymous medical data 46 (step ST160). In a case where the editing level is set to a second level or lower (N in step ST150), the pseudonym medical data 43 is maintained without performing the anonymization. In the second editing processing section 26, an accuracy reducing process on the pseudonym medical data 43 is performed according to the editing level (step ST170). In a case where the editing level is set to the second level or the fourth level (Y in step ST170), the accuracy reducing process is performed, and the health data in which an accuracy is reduced is generated (step ST180). In a case where the editing level is set to a first level or a third level (N in step ST180), the accuracy of each piece of health data is maintained without performing the accuracy reducing process. The medical data after the second editing process is output to the medical data using apparatus 13 via the output controller 29 (step ST190).

With the above contents, by determining the editing content of the medical data based on the output destination information, it is possible to utilize the data in accordance with the laws and regulations of each country, the safety of personal information, and an intention of a data user.

A modification example of the first embodiment will be described. In the first embodiment, a case where the medical data management apparatus 11, the medical data provision apparatus 12, and the medical data using apparatus 13 are implemented as individual apparatuses in the medical data management system 10 has been described, but in a case where editing of the personally identifiable medical data 40 is performed by a facility of the personally identifiable medical data 40 without being entrusted to a certified business operator, the functions of the medical data management apparatus 11 and the medical data provision apparatus 12 may be implemented by the same apparatus. For example, the function of the medical data management apparatus 11 is implemented in the medical data provision apparatus 12.

### Second Embodiment

In a second embodiment, an aspect in which a function of a medical data management system 60 is implemented by a facility on a medical data provision side and a facility on a medical data use side will be described. Instead of the medical data management apparatus 11, a first editing process is performed in the facility on the medical data provision side, and a second editing process is performed in the facility on the medical data use side. Since the other points are the same as those in the first embodiment, the description thereof will be omitted.

The medical data management apparatus 11, the medical data provision apparatus 12, and the medical data using apparatus 13 constituting the medical data management system 10 in the first embodiment include a state of being included in different facilities or companies, such as belonging to corporations different from each other. On the other hand, in some countries, there are restrictions such as medical data being utilized only in the own facility, so in that case, all the medical data are provided in the facility owned by the same corporation. However, since it is necessary to protect personal identification information even within the same corporation, the medical data using apparatus acquires at least pseudonymized medical data. Hereinafter, a medical data management system in a case where medical data is used by the same corporation will be described.

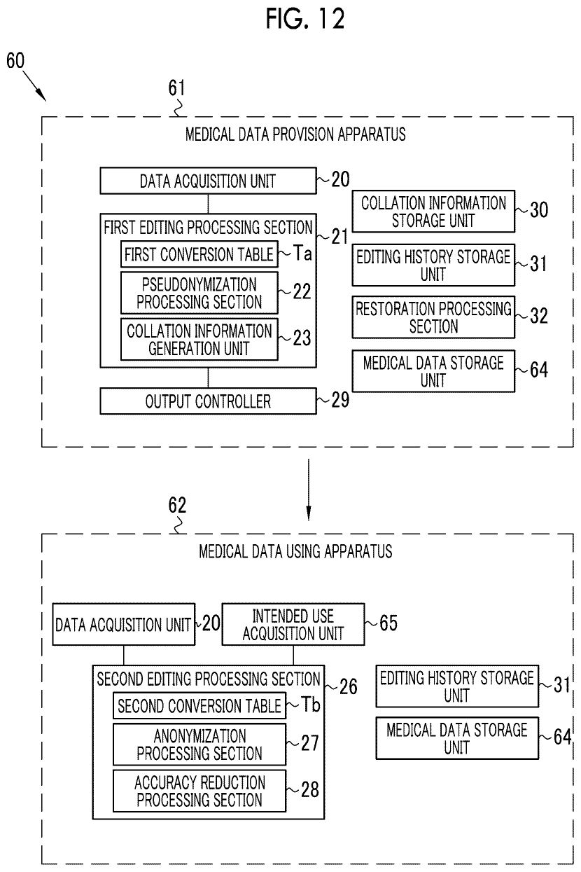

As illustrated in FIG. 12, the medical data management system 60 is configured with a communicable medical data provision apparatus 61 and a communicable medical data using apparatus 62, the medical data provision apparatus 61 has a function of outputting medical data on which a first editing process is performed, and the medical data using apparatus 62 has a function of using the medical data on which a second editing process is performed. The medical data provision apparatus 61 includes the data acquisition unit 20, the first editing processing section 21, the output controller 29, the collation information storage unit 30, the editing history storage unit 31, the restoration processing section 32, and a medical data storage unit 64, and the first editing processing section 21 implements the functions of the first conversion table Ta, the pseudonymization processing section 22, and the collation information generation unit 23. In addition, the medical data using apparatus 62 includes the data acquisition unit 20, the second editing processing section 26, the editing history storage unit 31, the medical data storage unit 64, and an intended use acquisition unit 65, and the second editing processing section 26 implements the functions of the second conversion table Tb, the anonymization processing section 27, and the accuracy reduction processing section 28.

The medical data storage unit 64 stores the edited and generated pseudonym medical data or anonymous medical data. The intended use acquisition unit 65 acquires information on the use of the medical data in the medical data using apparatus 62, and transmits the information to the second editing processing section 26. In addition, in a case where there is no particular description, each function other than the medical data storage unit 64 and the intended use acquisition unit 65 has the same function as in the first embodiment, and thus the description thereof will be omitted. Since affiliation information is information on the medical data using apparatus 62 that performs the second editing process, the data acquisition unit 20 or the second editing processing section 26 may automatically acquire the affiliation information.

The medical data provision apparatus 61 and the medical data using apparatus 62 are computers such as a personal computer or a workstation on which each application program for implementing predetermined functions is installed. The computer includes a central processing unit (CPU) which is a processor, a memory, a storage, and the like, and implements various functions by a program or the like stored in a storage.

The medical data provision apparatus 61 and the medical data using apparatus 62 may be provided in different facilities as long as the medical data provision apparatus 61 and the medical data using apparatus 62 belong to the same institution (the same corporation). The medical data provision apparatus 61 is a server that implements a function of the first editing process, and is a facility on a provision side of the medical data. In addition, the medical data using apparatus 62 is a server that implements a function of the second editing process, and is a facility on a use side of the medical data.

In the second embodiment, the facility that performs the first editing process is a facility on the provision side of the medical data, the facility that performs the second editing process is a facility on the use side of the medical data, and the step of transmitting the medical data after the first editing process from the server of the facility on the provision side of the medical data to the server of the facility on the use side of the medical data is included.

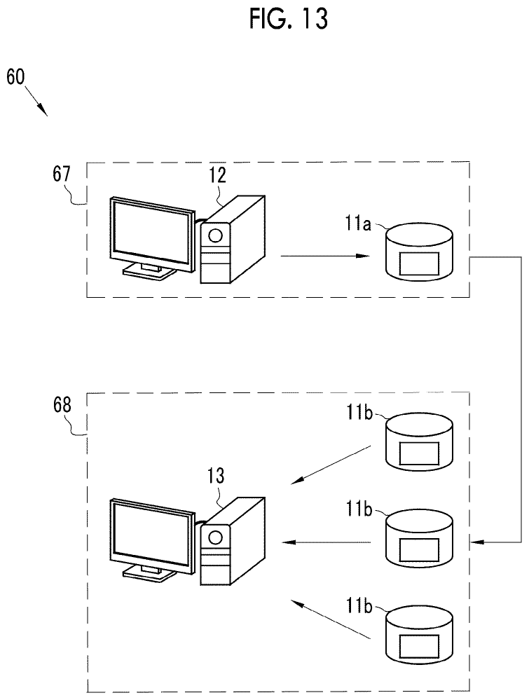

As illustrated in FIG. 13, a modification example of the second embodiment will be described. The medical data management system 60 is configured with a medical data providing facility 67 including the medical data provision apparatus 12 and a medical data management apparatus 11a which is a server implementing at least the function of the first editing process, and a medical data using facility 68 including the medical data using apparatus 13 and a medical data management apparatus 11b which is a server implementing at least the function of the second editing process. The medical data provision apparatus 12 and the medical data using apparatus 13 are the same as those in the first embodiment. It is preferable that a plurality of medical data management apparatuses 11b having a second editing processing section are provided differently depending on the newness of the version, the processing capacity of the apparatus, the type of anonymization software, or the like. For example, efficiency such as time saving can be achieved by selecting an appropriate medical data management apparatus 11b from among the plurality of medical data management apparatuses 11b according to the editing level.

It is preferable that the medical data provision apparatus 12 and the medical data management apparatus 11a are provided in the same facility, and it is preferable that the medical data using apparatus 13 and the medical data management apparatus 11b are provided in the same facility. The pseudonym medical data 43 generated by the medical data management apparatus 11a is transmitted to the medical data management apparatus 11b, and the second editing process is performed. The functions of the medical data management apparatuses 11a and 11b may be the same as the functions of the medical data management apparatus 11 in the first embodiment.

In the embodiment described above, the hardware structures of processing sections (processing units) that execute various types of processing, such as a central control unit (not illustrated), the data acquisition unit 20, the first editing processing section 21, the output destination information acquisition unit 25, the second editing processing section 26, the output controller 29, the collation information storage unit 30, the editing history storage unit 31, the restoration processing section 32, the medical data storage unit 64, and the intended use acquisition unit 65, are various processors as described below. The various processors include a central processing unit (CPU) that is a general-purpose processor which functions as various processing sections by executing software (programs), a programmable logic device (PLD) that is a processor of which a circuit configuration can be changed after manufacturing, such as a field programmable gate array (FPGA), a dedicated electrical circuit that is a processor having a circuit configuration exclusively designed to execute various types of processing, and the like.

One processing section may be configured with one of the various processors or may be configured with a combination of two or more processors of the same type or different types (for example, a plurality of FPGAs or a combination of a CPU and an FPGA). In addition, a plurality of processing sections may be configured by one processor. As an example in which the plurality of processing sections are configured by one processor, first, there is a form in which one processor is configured by a combination of one or more CPUs and software and the processor functions as the plurality of processing sections, as represented by a computer of a client or a server. Second, there is a form in which a processor that implements functions of the entire system including the plurality of processing sections with one integrated circuit (IC) chip is used, as represented by a system on chip (SoC). As described above, the various processing sections are configured by using one or more of the various processors as the hardware structure.

Further, the hardware structure of these various processors is more specifically an electric circuit (circuitry) in a form in which circuit elements such as semiconductor elements are combined. In addition, a hardware structure of the storage unit is a storage device such as a hard disc drive (HDD) and a solid state drive (SSD).

### Explanation of References

10: medical data management system
11: medical data management apparatus
11a: medical data management apparatus
11b: medical data management apparatus
12: medical data provision apparatus
13: medical data using apparatus
20: data acquisition unit
21: first editing processing section
22: pseudonymization processing section
23: collation information generation unit
25: output destination information acquisition unit
26: second editing processing section
27: anonymization processing section
28: accuracy reduction processing section
29: output controller
30: collation information storage unit
31: editing history storage unit
32: restoration processing section
40: personally identifiable medical data
41: personally identifiable information
42: personally identifiable health data
43: pseudonym medical data
44: pseudonym information
45: pseudonym health data
45a: pseudonym health data
45b: pseudonym health data
46: anonymous medical data
47: anonymous information
48: anonymous health data
48a: anonymous health data
50: medical image
51: anonymous medical image
60: medical data management system
61: medical data provision apparatus
62: medical data using apparatus
64: medical data storage unit
65: intended use acquisition unit
67: medical data providing facility
68: medical data using facility
C: individual collation information
H: administrator
ST110 to ST190: step
Ta: first conversion table
Tb: second conversion table

What is claimed is:

1. A medical data management apparatus comprising a processor,
wherein the processor is configured to:
perform a first editing process on medical data including personal information and health data of an individual, the first editing process editing an item of the personal information that is capable of identifying the individual; and
perform a second editing process on the medical data after the first editing process in accordance with an editing level according to an output destination.

2. The medical data management apparatus according to claim 1,
wherein the medical data after the first editing process is restorable to the medical data before the first editing process, and
the editing level is a level at which medical data after the second editing process is restorable to medical data before the second editing process.

3. The medical data management apparatus according to claim 2,
wherein in a case where the second editing process is performed on the medical data after the first editing process at the editing level at which the medical data is restorable, the individual can be identified from the medical data by combining a plurality of pieces of health data after the second editing process.

4. The medical data management apparatus according to claim 3,
wherein the editing level is such that an item of low priority in the restorable medical data after the second editing process has a lower accuracy than that item in the medical data before the second editing process.

5. The medical data management apparatus according to claim 1,
wherein the medical data after the first editing process is restorable to the medical data before the first editing process, and
the editing level is a level at which medical data after the second editing process is non-restorable to medical data before the second editing process.

6. The medical data management apparatus according to claim 5,
wherein the editing level is such that an item of low priority in the non-restorable medical data after the second editing process has a lower accuracy than that item in the medical data before the second editing process.

7. The medical data management apparatus according to claim 1,
wherein the processor is configured to:
set the editing level with reference to a conversion table in which the output destination and the editing level are associated with each other.

8. The medical data management apparatus according to claim 7,
wherein the conversion table is set or updated by a user interface.

9. The medical data management apparatus according to claim 8,
wherein the editing level is switched between a restorable level and a non-restorable level according to an intended use of the output destination.

10. The medical data management apparatus according to claim 9,
wherein the processor is configured to:
in a case where the intended use is the same and the output source and the output destination of the medical data are in the same country, define that state as having a standard editing level,
set the editing level higher than the standard editing level in a state in which the output source and the output destination are in different countries; and
set the editing level lower than the standard editing level in a state in which the output source and the output destination are in the same country and are related institutions.

11. The medical data management apparatus according to claim 1,
wherein the processor is configured to:
in the second editing process, perform a process of reducing the accuracy of information, for an item indicating a rare disease.

12. The medical data management apparatus according to claim 1,
wherein in a case where the medical data includes a medical image, the personal information includes personal identification information determined for each item and personal image information for identifying the individual from the medical image.

13. A non-transitory computer readable medium for storing a medical data management program, wherein the medical data management program causing a computer to execute:
a function of a first editing process on medical data including personal information and health data of an individual, the first editing process editing an item of the personal information that is capable of identifying the individual; and
a function of a second editing process on medical data after the first editing process in accordance with an editing level according to an output destination.

14. A medical data management method comprising:
a step of a first editing process on medical data including personal information and health data of an individual, the first editing process editing an item of the personal information that is capable of identifying the individual; and
a step of a second editing process on medical data after the first editing process in accordance with an editing level according to an output destination.

15. The medical data management method according to claim 14,
wherein a facility that performs the first editing process is a facility on a provision side of the medical data, and a facility that performs the second editing process is a facility on a use side of the medical data, and
the method further comprises:
a step of transmitting the medical data, after the first editing process and before the second editing process, from a server of the facility on the provision side of the medical data to a server of the facility on the use side of the medical data.
